(19) United States
(12) Patent Application Publication
(10) Pub. No.: US 2024/0270675 A1
(43) Pub. Date: Aug. 15, 2024

(54) METHOD FOR PREPARING CARBOXYLIC ACID ESTER COMPOUND

(71) Applicant: SOOCHOW UNIVERSITY, Suzhou (CN)

(72) Inventors: Xiaobing WAN, Suzhou (CN); Yonggao ZHENG, Suzhou (CN); Suyan TAO, Suzhou (CN); Xingxing LI, Suzhou (CN); Xionglue CHENG, Suzhou (CN)

(21) Appl. No.: 18/037,550

(22) PCT Filed: Feb. 7, 2021
(86) PCT No.: PCT/CN2021/075896

§ 371 Date: Nov. 21, 2023

(30) Foreign Application Priority Data
Dec. 25, 2020 (CN) 202011566880.0

(51) Int. Cl. C07C 67/08

(52) U.S. Cl. CPC C07C 67/08; C07C 2531/02

(57) ABSTRACT

The present invention relates to a method for preparing a carboxylic acid ester compound. Under the catalysis of nitrite, a carboxylic acid reacts with an alcohol in air so as to obtain an ester compound; and the alcohol is ethanol, propanol or trifluoroethanol. The present invention has the advantages such as mild reaction conditions, rich source of raw materials, wide universality of reaction substrates, and simple operation, and can modify a series of carboxylic acids having medicinal properties and aliphatic carboxylic acids such as biologically active amino acids.

### TECHNICAL FIELD

The present invention relates to the field of carboxylic acid ester synthesis technology, in particular to a method for synthesizing carboxylic acid methyl ester compounds.

### BACKGROUND OF INVENTION

The currently known methods for synthesizing esters mostly have the following disadvantages: the pre-activation is required for the raw materials, the reaction conditions are strict, the substrates used are harmful to the environment, the catalysts used are precious metals or heavy metals, which are expensive, the range of substrates is narrow, and atomic economy is poor; more importantly, due to the fact that general drug molecules have multiple functional groups, including sensory functional groups, while traditional esterification methods have poor tolerance to functional groups, the traditional esterification methods are generally not suitable for the esterification of drug molecules. For example, (1) The esterification of carboxylic acids and alcohols catalyzed by acids is a classic method for synthesizing esters. However, acids can cause damage to equipment and are harmful to the environment. At the same time, this method is not applicable when there are components sensitive to acids. (Reference: E. Emmet Reid; Ind. Eng. Chem; 1948, 40, 1596-1601; Junzo Otera; Chem. Rev. 1993, 93. 1449-1470).
(2) Debasis Manna et al. reported a method of generating carboxylic acid esters using carboxylic acids and alcohols as substrates in 2013. It used the heavy metal of zinc as an acid catalyst, and it used triphenyl phosphine and iodine in the reaction, which made the reaction more complex and produced by-products such as phosphorus oxide and hydrogen iodide, making the purification of the reaction more difficult. (Reference: Debasis Manna; J. Org. Chem. 2013, 78, 2386-2396).
(3) In 2013, Yasuhiro Uozumi et al. reported on a method for preparing carboxylic acid esters. The polymeric acid catalyst used in this method was not a commercial reagent and required further preparation. It was not practical for acid sensitive substances and had a narrow range of substrates. (Reference: Yasuhiro Uozumi; Org. Lett. 2013, 15, 5798-5801).
(4) Asit K. Chakraborti et al. reported on a method for preparing methyl carboxylate in 1999. However, the methylation reagents used in this method had high toxicity and were harmful to the environment and people; at the same time, this method used strong alkali. This limited the range of substrates, made the operation relatively cumbersome, and the atomic economy of the reaction was not high, so it caused serious pollution. (Reference: Asit K. Chakraborti; J. Org. Chem. 1999, 64, 8014-8017).
(5) Shannon S. Stahl et al. reported on a method for preparing carboxylic acid esters in 2017. This method used precious metal palladium catalysts, and it was expensive and had relatively complex reaction conditions. (Reference: Shannon S. Stahl; J. Am. Chem. Soc. 2017, 139, 1690-1698).
(6) Aiwen Lei et al. also reported on a method for preparing carboxylic acid esters in 2012. However, its reaction required expensive and complex palladium containing catalysts, and the reaction conditions were relatively complex, with a narrow range of substrates. The raw materials used were prone to decompose, and the price was generally several times that of the corresponding carboxylic acid. (Reference: Aiwen Lei; Angew. Chem. Int. Ed. 2012, 51, 1-6).

Based on the above, it is particularly important to develop an esterification method that includes but is not limited to the carboxylic acid of drug molecules.

### Technical Problems

To solve the above technical problems, the purpose of the present invention is to provide a method for preparing carboxylic acid ester compounds, and it has mild reaction conditions; rich sources of raw materials; wide universality of reaction substrates; and the advantages of simple operation to modify a series of carboxylic acids with drug properties and fatty carboxylic acid esters such as biologically active amino acids.

### Technical Solution

The present invention discloses a method for preparing carboxylic acid ester compounds. In the presence of nitrous acid ester, carboxylic acid compound and alcohol are used as raw materials to react and prepare carboxylic acid ester compounds; and the alcohol is ethanol, propanol, or trifluoroethanol.

The present invention discloses the application of nitrous acid ester in catalyzing the reaction of carboxylic acid compound and alcohol to prepare carboxylic acid ester compounds; and the alcohol is ethanol, propanol, or trifluoroethanol.

The method for preparing carboxylic acid ester compounds of the present invention is as follows: the nitrous acid ester, carboxylic acid compound and alcohol are added into a reaction tube in sequence in the air; then the reaction is carried out under the condition of 60-80° C. for 30-60 hours to obtain the carboxylate ester compounds.

The general formula of the carboxylic acid compound is:

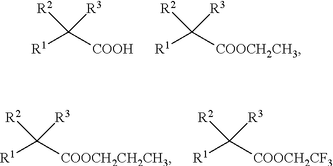

The general formula of the carboxylic acid compound is:

In the above formula, R¹ is selected from one of hydrogen, C1-C12 alkyl, alkoxy, phenyl, benzyl, substituent phenyl, thienyl, indolyl, phenolic group, naphthyl, biphenyl, or amide groups; R² is selected from one of hydrogen, methyl, methylene, ethyl, isopropyl, hydroxyl, hydroxymethyl, or phenyl; R³ is selected from one of hydrogen, methyl, methylene, ethyl, isopropyl, propyl, butyl, or phenyl; the substituents on the substituent phenyl are selected from one or more of hydrogen, methyl, methoxy, hydroxyl, nitro, phenyl, acetylamino, fluorine, chlorine, bromine, iodine, etc.; the nitrous acid ester is one or more of isopropyl nitrite, butyl nitrite, isobutyl nitrite, and tert-butyl nitrite, preferably tert-butyl nitrite (^{t}BuONO).

Furthermore, the molar ratio of the carboxylic acid compound to the nitrous acid ester is 10:5-20; preferably, the molar ratio of carboxylic acid compound to nitrous acid ester is 1:1.

Furthermore, the dosage ratio of carboxylic acid compound and alcohol is 0.5 mmol:2 mL.

Furthermore, the reaction time is 30-60 hours, preferably 48 hours; the reaction temperature is 60-80° C., preferably 80° C.

Furthermore, the reaction is carried out in the air.

Furthermore, after the reaction is completed, the quenching reaction is carried out with sodium thiosulfate to routinely separate carboxylic ester compound. For example, after the quenching reaction, the product is extracted with ethyl acetate, removed from the solvent and adsorbed on silica gel, and then subject to column chromatography to obtain the carboxylic ester compound.

### Beneficial Effects

### The Present Invention has at Least the Following Advantages:

1. The reaction substrates used in the present invention are commercially available and have good prospects for pharmaceutical and industrial applications.
2. In the present invention, the reaction can take place without the presence of additives such as metals, strong bases, and strong acids, meeting the eco-friendly requirements.
3. The present invention has high atomic economy and a byproduct of water; the reaction system is simple and has a wide range of substrates, good functional group compatibility, mild reaction conditions, and convenient post-processing operations, and it solves the problems of existing synthesis methods.
4. It can easily synthesize a series of carboxylic acids with drug properties and fatty carboxylic acid esters such as biologically active amino acids.

The above description is only an overview of the technical solution of the present invention. In order to have a clearer understanding of the technical means of the present invention and to implement it according to the content of the specification, the following are preferred embodiments of the present invention and detailed explanations.

### EXAMPLES OF THE PRESENT INVENTION

The raw materials of the present invention are all existing commercially available products, and the specific preparation and testing methods are conventional methods. The present invention only uses nitrous acid ester, carboxylic acid compound, and alcohol as raw materials for reaction without the addition of other substances to prepare carboxylic acid esters in the air under mild conditions, solving the problem of metal or metal compound catalytic reaction in the present invention, and overcoming the problem that traditional esterification methods are not suitable for esterification of drug molecules; the following is the detailed description of the specific embodiments of the present invention with embodiments. The following embodiments are used to illustrate the present invention, but are not intended to limit its scope.

### Example 1

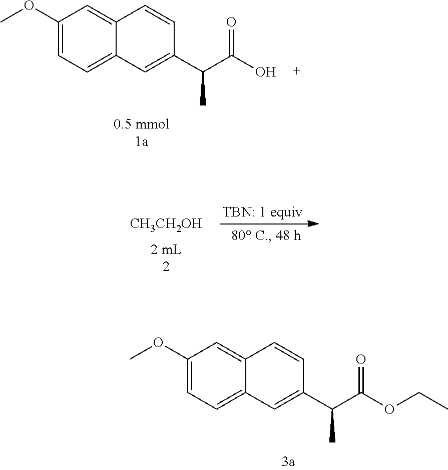 [SMILES: COc1ccc2cc([C@H](C)C(=O)O)ccc2c1 ; "CH3CH2OH" ; CCOC(=O)[C@@H](C)c1ccc2cc(OC)ccc2c1]

The drug molecule 1a (Naproxen) (0.5 mmol, 115.2 mg) and ethanol containing 1 equiv tert-butyl nitrite (2 mL ethanol, 0.5 mmol tert-butyl nitrite, the same meaning for the following embodiments) were added into the reaction tube in sequence; the reaction was carried out at 80° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3a, with a yield of 74% and separation yield. The main test data of the prepared product are as follows. Through analysis, it can be seen that the actual synthesized product is consistent with theoretical analysis.

¹H NMR (400 MHZ, CDCl₃) δ 7.75-7.68 (m, 3H), 7.45-7.43 (m, 1H), 7.19-7.12 (m, 2H), 4.22-4.09 (m, 2H), 3.91 (s, 3H), 3.86 (q, J=7.2 Hz, 1H), 1.60 (d, J=7.2 Hz, 3H), 1.22 (t, J=7.2 Hz, 3H); 13C NMR (100 MHz, CDCl₃) δ 174.5, 157.5, 135.7, 133.6, 129.1, 128.8, 127.0, 126.1, 125.8, 118.8, 105.5, 60.6, 55.1, 45.4, 18.5, 14.1; HRMS (ESI-TOF): Anal. Calcd. For C₁₆H₁₈O₃+Na⁺: 281.1148, Found: 281.1149; IR (neat, cm⁻¹): ν 3060, 2981, 2939, 1728, 1590, 1456, 1372, 1264, 1173, 1027, 856.

When the tert-butyl nitrite was replaced with the same molar amount of tert-butyl hydrogen peroxide, and the remaining kept unchanged, the product yield was less than 5%.

### Example 2

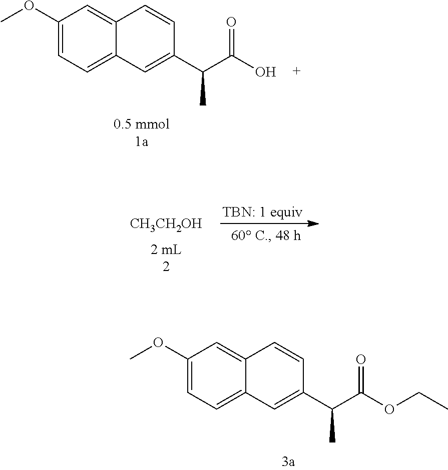 [SMILES: COc1ccc2cc([C@H](C)C(=O)O)ccc2c1 ; "CH3CH2OH" ; CCOC(=O)[C@@H](C)c1ccc2cc(OC)ccc2c1]

The drug molecule 1a (Naproxen) (0.5 mmol, 115.2 mg) and ethanol containing 1 equiv tert-butyl nitrite were added into the reaction tube in sequence; the reaction was carried out at 60° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3a, with a yield of 60% and separation yield. The main test data of the prepared product are as follows. Through analysis, it can be seen that the actual synthesized product is consistent with theoretical analysis.

### Example 3

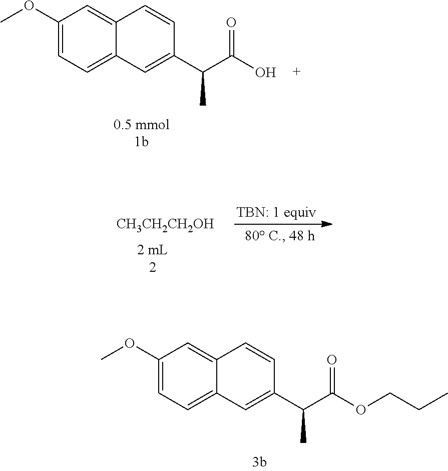 [SMILES: COc1ccc2cc([C@H](C)C(=O)O)ccc2c1 ; "CH3CH2CH2OH" ; CCCOC(=O)[C@@H](C)c1ccc2cc(OC)ccc2c1]

The drug molecule 1b (Naproxen) (0.5 mmol, 115.2 mg) and propanol containing 1 equiv tert-butyl nitrite were added into the reaction tube in sequence; the reaction was carried out at 80° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3b, with a yield of 72% and separation yield. The main test data of the prepared product are as follows. Through analysis, it can be seen that the actual synthesized product is consistent with theoretical analysis.

¹H NMR (400 MHZ, CDCl₃) δ 7.74-7.66 (m, 3H), 7.44-7.41 (m, 1H), 7.17-7.10 (m, 2H), 4.04 (t, J=6.7 Hz, 2H), 3.91 (s, 3H), 3.86 (q, J=7.2 Hz, 1H), 1.63-1.58 (m, 5H), 0.86 (t, J=7.4 Hz, 3H); 13C NMR (100 MHz, CDCl₃) δ 174.7, 157.6, 135.9, 133.6, 129.3, 128.9, 127.0, 126.3, 125.9, 118.9, 105.6, 66.3, 55.3, 45.5, 21.9, 18.5, 10.3; HRMS (ESI-TOF): Anal. Calcd. For C₁₇H₂₀O₃+Na⁺: 295.1305, Found: 295.1323; IR (neat, cm⁻¹): ν 2967, 2935, 1724, 1605, 1461, 1262, 1181, 858, 813.

### Example 4

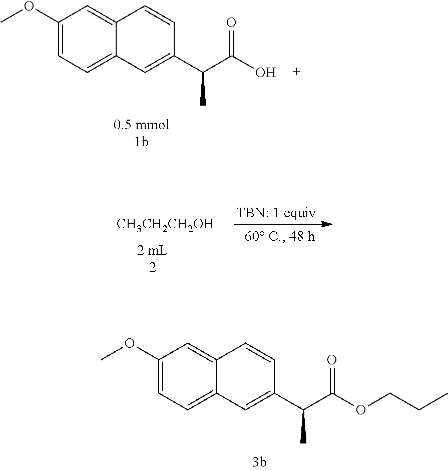 [SMILES: COc1ccc2cc([C@H](C)C(=O)O)ccc2c1 ; "CH3CH2CH2OH" ; CCCOC(=O)[C@@H](C)c1ccc2cc(OC)ccc2c1]

The drug molecule 1b (Naproxen) (0.5 mmol, 115.2 mg) and propanol containing 1 equiv tert-butyl nitrite were added into the reaction tube in sequence; the reaction was carried out at 60° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3b, with a yield of 37% and separation yield. The main test data of the prepared product are as follows. Through analysis, it can be seen that the actual synthesized product is consistent with theoretical analysis.

### Example 5

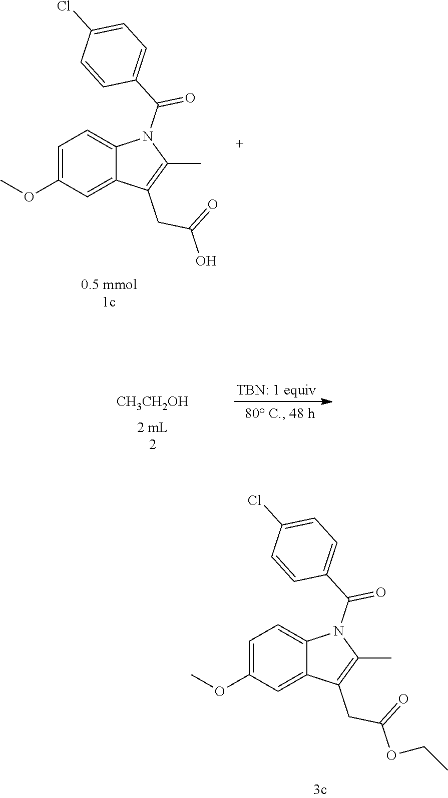 [SMILES: COc1ccc2c(c1)c(CC(=O)O)c(C)n2C(=O)c1ccc(Cl)cc1 ; "CH3CH2OH" ; CCOC(=O)Cc1c(C)n(C(=O)c2ccc(Cl)cc2)c2ccc(OC)cc12]

The drug molecule 1c (Indomethacin) (0.5 mmol, 178.9 mg) and ethanol containing 1 equiv tert-butyl nitrite were added into the reaction tube in sequence; the reaction was carried out at 80° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3c, with a yield of 60% and separation yield. The main test data of the prepared product are as follows. Through analysis, it can be seen that the actual synthesized product is consistent with theoretical analysis.

¹H NMR (400 MHZ, CDCl₃) δ 7.69-7.62 (m, 2H), 7.50-7.42 (m, 2H), 6.97 (d, J=2.5 Hz, 1H), 6.88 (d, J=9.0 Hz, 1H), 6.68-6.65 (m, 1H), 4.16 (q, J=7.1 Hz, 2H), 3.83 (s, 3H), 3.65 (s, 2H), 2.38 (s, 3H), 1.26 (t, J=7.1 Hz, 3H); 13C NMR (100 MHZ, CDCl₃) δ 170.8, 168.2, 156.0, 139.1, 135.8, 133.9, 131.1, 130.7, 130.6, 129.0, 114.9, 112.6, 111.6, 101.2, 60.9, 55.6, 30.3, 14.2, 13.3; HRMS (ESI-TOF): Anal. Calcd. For C₂₁H₂₀³⁵ClNO₄+Na⁺: 408.0973, Found: 408.0950. Anal. Calcd. For C₂₁H₂₀³⁷ClNO₄+Na⁺: 410.0944, Found: 410.0947; IR (neat, cm⁻¹): ν 2978, 2929, 1727, 1673, 1602, 1466, 1358, 1321, 1173, 1035, 912, 802.

### Example 6

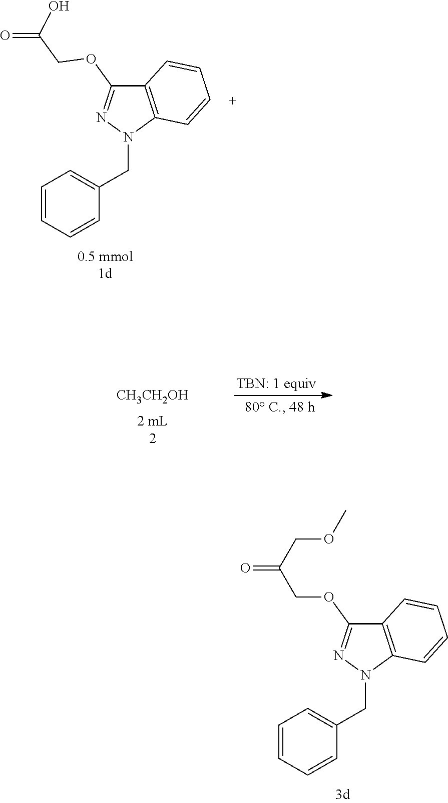 [SMILES: O=C(O)COc1nn(Cc2ccccc2)c2ccccc12 ; "CH3CH2OH" ; COCC(=O)COc1nn(Cc2ccccc2)c2ccccc12]

The drug molecule 1d (Bendazac) (0.5 mmol, 178.9 mg) and ethanol containing 1 equiv tert-butyl nitrite were added into the reaction tube in sequence; the reaction was carried out at 80° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3d, with a yield of 71% and separation yield. The main test data of the prepared product are as follows. Through analysis, it can be seen that the actual synthesized product is consistent with theoretical analysis.

¹H NMR (400 MHZ, CDCl₃) δ 7.73 (d, J=8.1 Hz, 1H), 7.32-7.19 (m, 4H), 7.17-7.09 (m, 3H), 7.05-7.01 (m, 1H), 5.33 (s, 2H), 4.98-4.92 (m, 2H), 4.21 (q, J=7.1 Hz, 2H), 1.22 (t, J=7.1 Hz, 3H); 13C NMR (100 MHz, CDCl₃) δ 168.8, 154.8, 141.7, 137.3, 128.5, 127.4, 126.9, 120.1, 119.3, 112.4, 108.8, 65.5, 61.0, 52.2, 14.0; HRMS (ESI-TOF): Anal. Calcd. For C₁₈H₁₈N₂O₃+Na⁺: 333.1210, Found: 333.1225; IR (neat, cm⁻¹): ν 2977, 2932, 1752, 1684, 1615, 1530, 1495, 1452, 1198, 1145, 1063, 737.

### Example 7

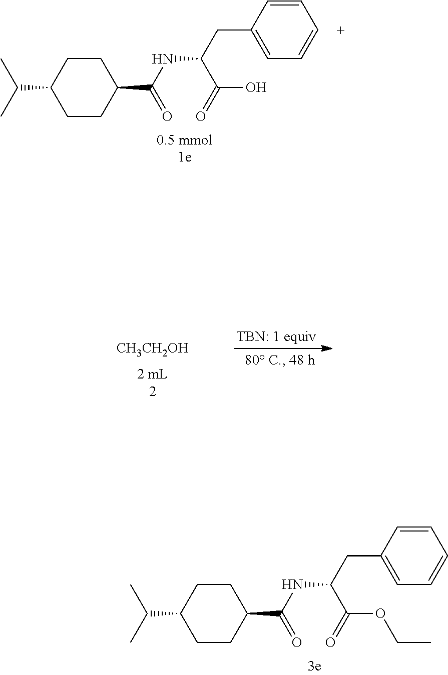 [SMILES: CC(C)[C@H]1CC[C@H](C(=O)N[C@H](Cc2ccccc2)C(=O)O)CC1 ; "CH3CH2OH" ; CCOC(=O)[C@@H](Cc1ccccc1)NC(=O)[C@H]1CC[C@H](C(C)C)CC1]

The drug molecule 1e (Nateglinide) (0.5 mmol, 158.8 mg) and ethanol containing 1 equiv tert-butyl nitrite were added into the reaction tube in sequence; the reaction was carried out at 80° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3e, with a yield of 94% and separation yield. The main test data of the prepared product are as follows. Through analysis, it can be seen that the actual synthesized product is consistent with theoretical analysis.

¹H NMR (400 MHZ, CDCl₃) δ 7.32-7.18 (m, 3H), 7.13-7.06 (m, 2H), 6.03 (d, J=7.7 Hz, 1H), 4.88-4.83 (m, 1H), 4.16 (q, J=7.2 Hz, 2H), 3.15 (dd, J=13.8, 5.9 Hz, 1H), 3.08 (dd, J=13.8, 5.8 Hz, 1H), 2.05-1.97 (m, 1H), 1.91-1.82 (m, 2H), 1.81-1.72 (m, 2H), 1.46-1.33 (m, 3H), 1.24 (t, J=7.2 Hz, 3H), 1.10-0.90 (m, 3H), 0.85 (d, J=8.8 Hz, 6H); ¹³C NMR (100 MHZ, CDCl₃) δ 175.4, 171.6, 135.9, 129.2, 128.3, 126.8, 61.3, 52.6, 45.3, 43.1, 37.7, 32.6, 29.6, 29.3, 28.84, 28.75, 19.6, 14.0; HRMS (ESI-TOF): Anal. Calcd. For C₂₁H₃₁NO₃+Na⁺: 368.2196, Found: 368.2187; IR (neat, cm⁻¹): ν 3310, 2976, 2931, 2868, 1724, 1641, 1539, 1445, 1279, 1180, 697.

### Example 8

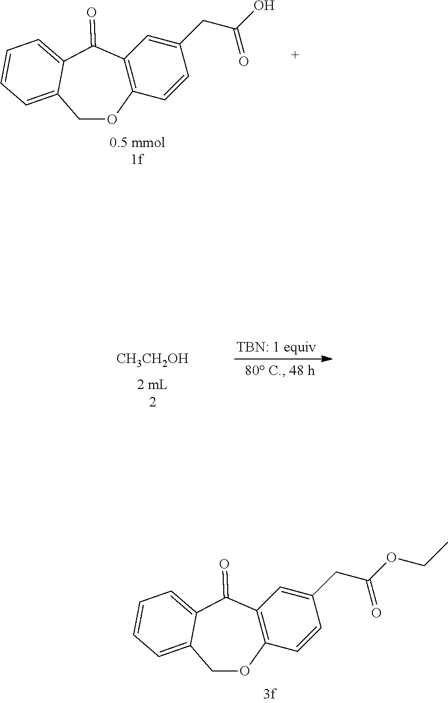 [SMILES: O=C(O)Cc1ccc2c(c1)C(=O)c1ccccc1CO2 ; "CH3CH2OH" ; CCOC(=O)Cc1ccc2c(c1)C(=O)c1ccccc1CO2]

The drug molecule If (Isoxepac) (0.5 mmol, 134.2 mg) and ethanol containing 1 equiv tert-butyl nitrite were added into the reaction tube in sequence; the reaction was carried out at 80° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3f, with a yield of 98% and separation yield. The main test data of the prepared product are as follows. Through analysis, it can be seen that the actual synthesized product is consistent with theoretical analysis.

¹H NMR (400 MHZ, CDCl₃) δ 8.10 (d, J=2.3 Hz, 1H), 7.87-7.84 (m, 1H), 7.52-7.48 (m, 1H), 7.45-7.38 (m, 2H), 7.33-7.28 (m, 1H), 6.99 (d, J=8.4 Hz, 1H), 5.13 (s, 2H), 4.14 (q, J=7.1 Hz, 2H), 3.60 (s, 2H), 1.24 (t, J=7.1 Hz, 3H); 13C NMR (100 MHz, CDCl₃) δ 190.6, 171.2, 160.3, 140.3, 136.2, 135.4, 132.6, 132.3, 129.3, 129.1, 127.8, 127.6, 125.0, 120.9, 73.4, 60.8, 40.1, 14.0; HRMS (ESI-TOF): Anal. Calcd. For C₁₈H₁₆O₄+Na⁺: 319.0941, Found: 319.0947; IR (neat, cm⁻¹): ν 3059, 2981, 2957, 2924, 1733, 1654, 1612, 1490, 1300, 1176, 1008, 769.

### Example 9

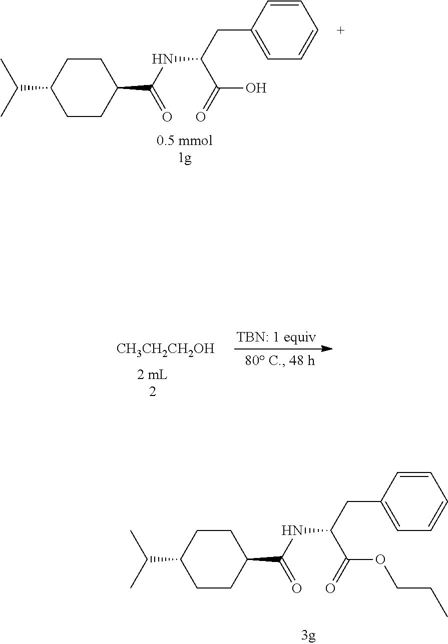 [SMILES: CC(C)[C@H]1CC[C@H](C(=O)N[C@H](Cc2ccccc2)C(=O)O)CC1 ; "CH3CH2CH2OH" ; CCCOC(=O)[C@@H](Cc1ccccc1)NC(=O)[C@H]1CC[C@H](C(C)C)CC1]

The drug molecule 1g (Nateglinide) (0.5 mmol, 158.8 mg) and propanol containing 1 equiv tert-butyl nitrite were added into the reaction tube in sequence; the reaction was carried out at 80° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3g, with a yield of 90% and separation yield. The main test data of the prepared product are as follows. Through analysis, it can be seen that the actual synthesized product is consistent with theoretical analysis.

¹H NMR (400 MHZ, CDCl₃) δ 7.30-7.20 (m, 3H), 7.12-7.06 (m, 2H), 6.01 (d, J=7.8 Hz, 1H), 4.90-4.85 (m, 1H), 4.12-4.06 (m, 1H), 4.06-4.01 (m, 2H), 3.15 (dd, J=13.8, 6.0 Hz, 1H), 3.08 (dd, J=13.8, 5.8 Hz, 1H), 2.04-1.97 (m, 1H), 1.91-1.83 (m, 2H), 1.78-1.75 (m, 2H), 1.69-1.58 (m, 2H), 1.47-1.33 (m, 3H), 1.10-0.94 (m, 3H), 0.91 (t, J=7.4 Hz, 3H), 0.85 (d, J=6.8 Hz, 6H); 13C NMR (100 MHz, CDCl₃) δ 175.5, 171.8, 135.9, 129.2, 128.3, 126.9, 66.9, 52.6, 45.3, 43.1, 37.8, 32.6, 29.6, 29.4, 28.86, 28.78, 21.7, 19.6, 10.2; HRMS (ESI-TOF): Anal. Calcd. For C₂₂H₃₃NO₃+H⁺: 360.2533, Found: 360.2530; IR (neat, cm⁻¹): ν 3301, 2975, 2927, 2858, 1724, 1638, 1548, 1444, 1286, 1182, 696.

### Example 10

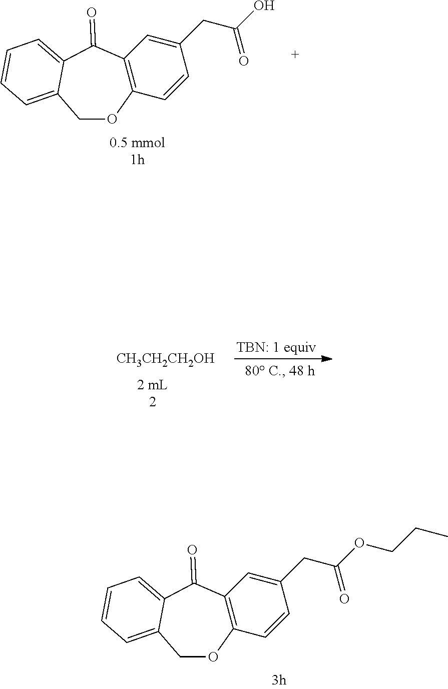 [SMILES: O=C(O)Cc1ccc2c(c1)C(=O)c1ccccc1CO2 ; "CH3CH2CH2OH" ; CCCOC(=O)Cc1ccc2c(c1)C(=O)c1ccccc1CO2]

The drug molecule 1 h (Isoxepac) (0.5 mmol, 134.2 mg) and propanol containing 1 equiv tert-butyl nitrite were added into the reaction tube in sequence; the reaction was carried out at 80° C. for 48 hours in the air; after the reaction was completed, sodium thiosulfate was added and stirred for quenching. The solvent was then removed by a rotary evaporator and adsorbed on silica gel. Finally, a mixed solvent of ethyl acetate and petroleum ether was used for column chromatography to obtain product 3 h, with a yield of 97% and separation yield. The main test data of the prepared product are as follows. Through analysis, it can be seen that the actual synthesized product is consistent with theoretical analysis.

¹H NMR (400 MHZ, CDCl₃) δ 8.10 (d, J=2.3 Hz, 1H), 7.87-7.84 (m, 1H), 7.53-7.49 (m, 1H), 7.46-7.38 (m, 2H), 7.32-7.30 (m, 1H), 6.99 (d, J=8.4 Hz, 1H), 5.13 (s, 2H), 4.04 (t, J=6.7 Hz, 2H), 3.62 (s, 2H), 1.68-1.58 (m, 2H), 0.90 (t, J=7.4 Hz, 3H); 13C NMR (100 MHZ, CDCl₃) δ 190.6, 171.3, 160.3, 140.3, 136.2, 135.4, 132.6, 132.3, 129.3, 129.1, 127.83, 127.65, 125.0, 120.8, 73.4, 66.4, 40.1, 21.8, 10.2; HRMS (ESI-TOF): Anal. Calcd. For C₁₉H₁₈O₄+H⁺: 333.1097, Found: 333.1111; IR (neat, cm⁻¹): ν 3060, 2976, 2879, 1730, 1648, 1599, 1489, 1298, 1171, 1139, 766.

The foregoing is only a preferred Example of the present invention and is not intended to limit the present invention, and it should be noted that for a person of ordinary skill in the art, several improvements and variations can be made without departing from the technical principles of the present invention, which should also be regarded as the scope of protection of the present invention. The method of the present invention has the advantages of abundant source of raw materials, easy operation, strong compatibility of functional groups, good universality of substrates, green and safe, and can modify a series of known drug molecules by methyl esterification, which is also a shortcut to develop and discover new drug molecules or physiologically active molecules.

1. A method for preparing a carboxylic acid ester compound, wherein in the presence of a nitrous acid ester, a carboxylic acid compound and an alcohol are used as starting materials to react and prepare the carboxylic acid ester compound; and the alcohol is ethanol, propanol, or trifluoroethanol.

2. The method for preparing the carboxylic acid ester compound according to claim 1, wherein a general formula of the carboxylic acid compound is: 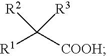
R¹ is selected from group consisting of hydrogen, C₁-C₁₂ alkyl, alkoxy, phenyl, benzyl, substituent phenyl, thienyl, indolyl, phenolic group, naphthyl, biphenyl, and an amide group; R² is selected from the group consisting of hydrogen, methyl, methylene, ethyl, isopropyl, hydroxyl, hydroxymethyl, and phenyl; R³ is selected from the group consisting of hydrogen, methyl, methylene, ethyl, isopropyl, propyl, butyl, and phenyl; substituents on the substituent phenyl are selected from one or more selected from the group consisting of hydrogen, methyl, methoxy, hydroxyl, nitro, phenyl, acetylamino, fluorine, chlorine, bromine, and iodine.

3. The method for preparing the carboxylic acid ester compound according to claim 1, wherein the nitrous acid ester is selected from the group consisting of isopropyl nitrite, butyl nitrite, isobutyl nitrite and tert-butyl nitrite.

4. The method for preparing the carboxylic acid ester compound according to claim 1, wherein a molar ratio of the carboxylic acid compound to the nitrous acid ester is 10:5-20.

5. The method for preparing the carboxylic acid ester compound according to claim 1, wherein a molar ratio of the carboxylic acid compound to the nitrous acid ester is 1:1.

6. The method for preparing the carboxylic acid ester compound according to claim 1, wherein a reaction time is 30-60 hours; a reaction temperature is 60-80° C.

7. The method for preparing the carboxylic acid ester compound according to claim 1, wherein the reaction is carried out in air.

8. A carboxylic acid ester compound prepared by the method for preparing carboxylic acid ester compounds according to claim 1.

9. An application of nitrous acid ester in catalyzing a reaction of carboxylic acid compound and an alcohol to prepare a carboxylic acid ester compound; and the alcohol is ethanol, propanol, or trifluoroethanol.

10. The application according to claim 9, wherein the reaction is free of a metals or a metal compound.
